(19) United States
(12) Patent Application Publication
(10) Pub. No.: US 2012/0189707 A1
(43) Pub. Date: Jul. 26, 2012

(54) Production Method For Cryopreserved Acellular Dermal Matrix, And Cryopreserved Acellular Dermal Matrix Produced Thereby

(75) Inventors: Wook Chun, Seoul (KR); Weon Ik Choi, Gyeonggi-do (KR); Man Seong Park, Seoul (KR); Geun Hyung Kim, Gwangju (KR); Jae Deuk Jung, Seoul (KR)

(73) Assignee: INDUSTRY ACADEMIC COOPERATION FOUNDATION HALLYM UNIVERSITY, Chuncheon, Gangwon-do (KR)

(21) Appl. No.: 13/392,596

(22) PCT Filed: Sep. 9, 2010
(86) PCT No.: PCT/KR2010/006146

§ 371 Date: Apr. 13, 2012

(30) Foreign Application Priority Data
Sep. 11, 2009 (KR) 10-2009-0085666

(51) Int. Cl. A61K 35/36; A61P 17/00

(52) U.S. Cl. 424/574

(57) ABSTRACT

The present invention relates to a production method for cryopreserved acellular dermal matrix and to cryopreserved acellular dermal matrix produced thereby, and more specifically it relates to a method in which a cryopreservation agent is made by adding sucrose to basic components consisting of glycerol and a basic solution and in which the resulting solution is used in the cryopreservation of skin tissue from which the cells in the epidermis and dermis have been removed, and relates to cryopreserved acellular dermal matrix produced thereby.

### TECHNICAL FIELD

The present invention relates to a method for preparing a cryopreserved acellular dermal matrix and a cryopreserved acellular dermal matrix prepared therefrom. More specifically, the present invention relates to a method in which a cryoprotectant is prepared by adding sucrose to basic constituents comprising glycerol and a basic solution, and then the resulting solution is used to subject skin tissue in which epidermis and cells in dermis are removed to a cryopreservation process and a cryopreserved acellular dermal matrix prepared therefrom.

### BACKGROUND ART

Skin is the largest organ, covering the entire human body, and has functions of preventing loss of body fluid, influx of toxic substances and microbes from the outside, and protecting the body from physical and chemical stimuli. In the case of a patient whose skin is seriously impaired by severe burns, injury, carcinoma excision, skin diseases and the like, a protective membrane is needed to prevent infection of impaired regions and the loss of body fluid, along with not leaving a scar at the impaired region and preventing serious shrinkage accompanied by the process of spontaneous cure. For regenerating impaired skin tissue, there are three methods of autograft in which a patient's own skin is transplanted, allograft in which the skin of another human being is transplanted and xenograft in which the skin of an animal is transplanted. Among them, autograft is the most ideal. However, when burnt areas are extensive, there is a limitation in the region from which skin tissue may be obtained, and the harvesting region can leave a new scar. Allograft plays a greater role in helping the movement of cells at the periphery of the impaired region and curing than permanent engraftment.

Specifically, in the case of a third-degree burn in which epidermis, dermis and subcutaneous layers are impaired, skin grafting is essentially required. At present, autograft is the most often used as skin grafting. However, harvesting autograft tissue creates a new injury, increasing patient's pain, time for complete recovery can be extended, and the economic burden is greater. In addition, when insufficient healthy regions remain—as with a severely burned patient—autograft cannot be applied or grafting operations should be performed repeatedly. To resolve the above problems, allograft using the skin of another person and xenograft using the skin of an animal such as a pig have been tried. However, other side effects as well as immunorejection often result.

In the case of burn surgeries which are most generally performed in domestic and foreign hospitals, the dead epidermis and dermis layers are removed and skin grafting is then carried out by using an acellular dermis in which the epidermis and cells in the dermis are removed from skin harvested from a corpse to avoid immunorejection. Cultured keratinocytes will then complete the entire skin thereon. Because such a completed skin includes basement membrane, it can play a role in protecting the body from external hazardous substances. However, such a skin graft is very expensive and has a problem in balancing supply and demand since most skin grafts are imported. An acellular dermis in which the epidermis and cells in the dermis are removed from skin harvested from a corpse to avoid immunorejection is usually used after freezing, for the convenience of storage. However, because collagen tissue in the acellular dermis is destroyed in the course of freezing, the acellular dermis matrix may be rapidly degraded after transplantation.

### DETAILED DESCRIPTION OF THE INVENTION

### Technical Problem

Therefore, the technical problem to be solved in the present invention is the provision of a new method for preparing a cryopreserved acellular dermal matrix which can efficiently increase stability of tissue and maintain extracellular matrix structure without impairment as compared with the conventional methods, when skin tissue for transplantation is processed.

### Solution to the Problem

To solve the above problems, the present invention provides a method for preparing a cryopreserved acellular dermal matrix comprising:

i) removing epidermis of allograft skin;

ii) removing cells in dermis;

iii) mixing glycerol, and a basic solution selected from a buffer solution and an animal cell culture medium;

iv) dissolving sucrose in the solution to a final concentration of 20 to 40% by weight to obtain a cryoprotectant;

v) penetrating the cryoprotectant into the skin from which epidermis and cells in dermis are removed; and

vi) freezing the cryoprotectant-penetrated skin in a controlled rate freezer.

The present invention also provides an autograft substitute comprising a cryopreserved acellular dermal matrix which is prepared by the above method.

Hereinafter, the present invention is described in detail.

In the present invention, epidermis and cells in dermis of allograft skin are removed to avoid immunorejection. The removal of epidermis and cells in dermis may be carried out according to various methods known in the art, and there is no special limitation thereto. The removal of epidermis may be carried out, for example, by treatment with enzymes such as trypsin, collagenase or dispase, or NaCl solution. The removal of cells in dermis may be carried out, for example, by treatment with Triton X100, Tween 20, Tween 40, Tween 60, Tween 80, SDS (sodium dodecylsulfate) and the like.

In the present invention, glycerol and a basic solution are used as basic constituents of a cryoprotectant. In the present invention, the basic solution refers to a solution which acts as a base for the preparation of the cryoprotectant, and a buffer solution which is used in treating animal cells or an animal cell culture medium may be used. In the present invention, the buffer solution—which is used in the treatment of animal cells—may be used without specific limitation. The example of the buffer solution includes, but is not limited to, PBS (phosphate buffered saline), TBS (Tris-buffered saline), citric acid buffer and the like. In the present invention, the animal cell culture medium used may be any medium known in the art. In the present invention, the example of the animal cell culture medium includes, but is not limited to, MEM (Minimum Essential Media), DMEM (Dulbecco's Modified Eagle Media), RPMI 1640, IMDM (Iscove's Modified Dulbecco's Media), Defined Keratinocyte-SFM (without BPE), Keratinocyte-SFN (with BPE), KnockOut D-MEM, AmnioMAX-II Complete Medium, AmnioMAX-C100 Complete Medium. In the present invention, glycerol and the basic solution may be preferably used in a mixing ratio of 0.5˜3.5:9, more preferably 0.8˜2:9, most preferably 1:9, based on weight. In the present invention, if the mixing ratio of glycerol is less than 0.5, there may be a problem of freezing damage in a freezing step. If the mixing ratio of glycerol is greater than 3.5, there may be a problem of the denaturation of tissue after freezing.

In the present invention, a cryoprotectant is prepared by dissolving sucrose in the solution in which glycerol and the basic solution are mixed to the final concentration of 20 to 40% by weight. In the present invention, when sucrose is added to the cryoprotectant, it plays a role in stabilizing and protecting cell membranes and cell membrane proteins from ice crystals formed in a freezing step. As a result, the stability of tissue of the cryopreserved acellular dermal matrix prepared according to the present invention can be improved. In addition, the optimal mixing ratio of glycerol, the basic solution and sucrose can further improve the stability of the dermal tissue and maintain the structure of extracellular matrix without impairment. In the present invention, if the concentration of sucrose is less than 20% by weight, the stability of the tissue may be decreased due to ice crystals formed in a freezing step. If the concentration of sucrose is greater than 40% by weight, the stability of the tissue may be deteriorated by sugar crystals formed in the tissue after freeze-drying due to high concentration of sugar ingredients. In the present invention, the cryoprotectant is preferably prepared by dissolving sucrose in the basic constituents-mixed solution to the final concentration of 25 to 35% by weight and most preferably 30% by weight.

In the present invention, the penetration of the cryoprotectant into skin tissue may be carried out according to conventional methods known in the art. Preferably, the cryoprotectant may be penetrated into the skin tissue in a low-temperature bath. Time needed for penetration may vary depending on the size of skin tissue and other factors. For example, the cryoprotectant may be penetrated into the skin tissue in a 4° C. low-temperature bath for about 6 to 24 hours.

In the present invention, the cryoprotectant-penetrated skin is frozen by using a controlled rate freezer. Use of the controlled rate freezer allows the skin tissue to be frozen at a desired rate. In the present invention, the freezing rate of skin with the controlled rate freezer is preferably −0.1° C. to −7° C. per minute, more preferably −0.5° C. to −5° C., still more preferably −0.8° C. to −3° C., and most preferably −1° C. per minute. In the present invention, if the freezing rate is less than −0.1° C., the skin tissue freezes too slowly. As a result, the tissue may be destroyed by the formation of large ice crystals at the exterior of the tissue since more solute inside of the tissue than outside causes a lowering of the freezing rate inside of the tissue. In addition, when the skin tissue is frozen, the temperature of cryoprotectant-penetrated skin is different from the chamber temperature of the controlled rate freezer. As a result, if latent heat of fusion is not controlled by an excessive freezing rate of −7° C. per minute due to rapid freezing from the region in which latent heat of fusion is generated at freezing to −80° C. which is the temperature where the movement of water molecules stops, the skin tissue may be damaged by the formation of ice crystals.

### Effects of the Invention

The cryopreserved acellular dermal matrix prepared according to the present invention can be efficiently used as a substitute for autograft since the stability of the tissue is high, and extracellular matrix and basement membrane are well maintained without impairment.

### BRIEF DESCRIPTION OF THE DRAWINGS

FIG. 1 is scanning electron microscope photographs of acellular dermal matrixes of Example and Comparative Example with 60× and 150× magnifications. (A: Comparative Example, 60×; B: Comparative Example, 150×; C: Example, 60×; D: Example, 150×).

FIG. 2 is optical microscope photographs of acellular dermal matrixes of Example and Comparative Example with 100× and 200× magnifications. (A: Comparative Example, 100×; B: Comparative Example, 200×; C: Example, 100×; D: Example, 200×).

FIG. 3 is a graph representing results of degradability measured by the treatment of cryopreserved acellular dermal matrixes which are processed with cryoprotectants comprising sucrose in the final concentration of 10, 15, 20, 25, 30, 35 and 40% by weight with collagenase. (P.C. (positive control): treatment of collagen powder with collagenase; N.C. (negative control): No treatment of collagenase; D.W.: distilled water).

### DESCRIPTION OF EMBODIMENTS

The present invention is explained in more detail with the following examples. However, it must be understood that the protection scope of the present invention is not limited to the examples.

Because human skin tissue harvested from a donor (cadaver) is prohibited from being used in an experiment, pig skin—which is the closest to human skin—is used for preparing ten (10) of both cryopreserved skins and glycerol-preserved skins according to the following methods of Example and Comparative Example.

### EXAMPLE

Cryopreserved skin was prepared with pig skin according to the following steps.

(1) Pig skin was washed with saline solution.

(2) The pig skin was cut at the size of 5×10 cm².

(3) The pig skin was immersed in 1M NaCl (Sigma, USA) solution.

(4) A 38° C. incubator (P-039, CoreTech, Korea) was prepared.

(5) The reaction of the pig skin immersed in 1M NaCl (Sigma, USA) solution was carried out in the 38° C. incubator with stirring for about 6 to 24 hours.

(6) Epidermis was removed by using forceps.

(7) The dermis from which epidermis has been removed was washed with phosphate buffered saline (pH 7.2, Gibco, USA).

(8) The washed dermis was immersed in 0.1% SDS and reacted with stirring at room temperature for 1 hour to remove cells from the dermis.

(9) The dermis from which cells have been removed was washed with phosphate buffered saline.

(10) Glycerol (Sigma, USA) and phosphate buffered saline were mixed in the weight ratio of 1:9.

(11) Sucrose (Sigma, USA) was added to the solution of step (10) as the final concentration of 30% by weight and dissolved to obtain a cryoprotectant.

(12) A low-temperature bath (P-039, CoreTech, Korea) was set at 4° C.

(13) The pig skin of step (9) was put in the 4° C. low-temperature bath, and then the cryoprotectant was penetrated into the pig skin for 12 hours.

(14) The penetration-completed pig skin was put in a polyamide bag (CryoBag™, Origen, USA).

(15) A controlled rate freezer (14S-A, SY Lab, USA) was prepared.

(16) The polyamide bag of step (14) was put in the controlled rate freezer and frozen to −150° C. at the rate of −1° C. per minute.

(17) After freezing, the polyamide bag was kept frozen in a dry shipper until analysis experiments.

### Comparative Example

A freeze-dried skin was prepared with pig skin according to the following steps.

(1) Pig skin was washed with saline solution.

(2) The pig skin was cut at the size of 5×10 cm².

(3) The pig skin was immersed in 1M NaCl (Sigma, USA) solution.

(4) A 38° C. incubator (P-039, CoreTech, Korea) was prepared.

(5) The reaction of the pig skin immersed in 1M NaCl (Sigma, USA) solution was carried out in the 38° C. incubator with stirring for about 6 to 24 hours.

(6) Epidermis was removed by using forceps.

(7) The dermis from which epidermis has been removed was washed with phosphate buffered saline (pH 7.2, Gibco, USA).

(8) The washed dermis was immersed in 0.1% SDS and reacted with stirring at room temperature for 1 hour to remove cells from the dermis.

(9) The dermis from which cells have been removed was washed with phosphate buffered saline.

(10) Glycerol (Sigma, USA) and phosphate buffered saline were mixed in the weight ratio of 1:9 to obtain a cryoprotectant.

(11) A low-temperature bath (P-039, CoreTech, Korea) was set at 4° C.

(12) The pig skin of step (9) was put in the 4° C. low temperature bath, and then the cryoprotectant was penetrated into the pig skin for 12 hours.

(13) The penetration-completed pig skin and 50 ml of the cryoprotectant were put in a Tyvek bag (Korea C & S Co., Ltd., Korea).

(14) A freezing dryer (Genesis 25XL, VirTis, USA) was prepared.

(15) The Tyvek bag of step (13) was put in the freezing dryer and frozen to −70° C. at the rate of −1° C. per minute, and then dried under the vacuum of 5 torr for 24 hours to obtain a freeze-dried acellular dermis matrix.

(16) After freeze-drying, the freeze-dried acellular dermis matrix was sterilized in an E.O. gas sterilizer (HS-4313EO, HanShin Medical Co., Ltd., Korea).

(17) The sterilized, freeze-dried acellular dermis matrix was sealed in an aluminum bag and stored at room temperature until analysis experiments.

### Experimental Example 1

### Histological Examination

The pig skins prepared according to the above Example and Comparative Example were stained with H & E (hematoxylin & eosin) as follows:

(1) A paraffin block was cut with 4 μm thickness and dried to obtain a paraffin section.

(2) For deparaffinization, after conducting xylene treatment of 5 minutes three times, 100% ethanol treatment of 2 minutes two times, 90% ethanol treatment of 1 minute one time, 80% ethanol treatment of 1 minute one time and 70% ethanol treatment of 1 minute one time, the section was rinsed in running water for 10 minutes.

(3) After staining with hematoxylin for 10 minutes, the section was rinsed in running water for 3 minutes. Then, after staining with eosin for 10 minutes, the section was rinsed in running water until no eosin was detected in the rinse water. After conducting 70% ethanol treatment of 1 second ten times, 80% ethanol treatment of 1 second ten times, 90% ethanol treatment of 1 second ten times, 100% ethanol treatment of 1 minute two times and xylene treatment of 3 minutes three times, the section was mounted with a mounting solution.

The scanning electron microscope observation of the pig skins prepared according to the above Example and Comparative Example was carried out as follows:

(1) A specimen was pre-fixed with 2.5% glutaraldehyde solution (fixative solution) for 2 hours, washed with 0.1M phosphate buffered saline and post-fixed with 1% OsO₄ solution.

(2) The fixed specimen was hydrated and substituted through a series of increased ethanol concentration, and then the specimen was frozen and fractured in −190° C. liquid nitrogen to expose the cross section, and completely dried by using a critical point dryer (HCP-2).

(3) The specimen was fixed at an aluminum stub (specimen mount) with the fractured surface upward, and metal coated with Pt—Pd at about 10 mm thickness by using a metal ion coating system (E-1030 ion sputter).

(4) The specimen was observed and photographed with a scanning electron microscope (Hitachi S-4700, Japan).

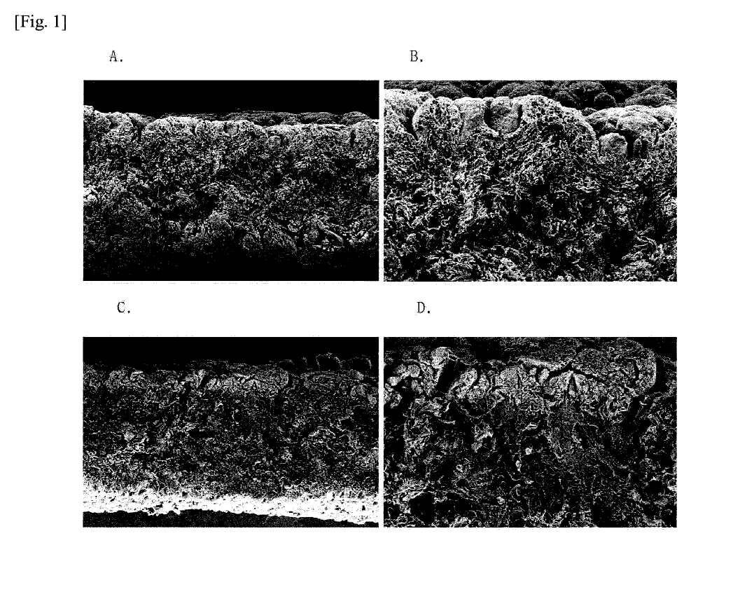

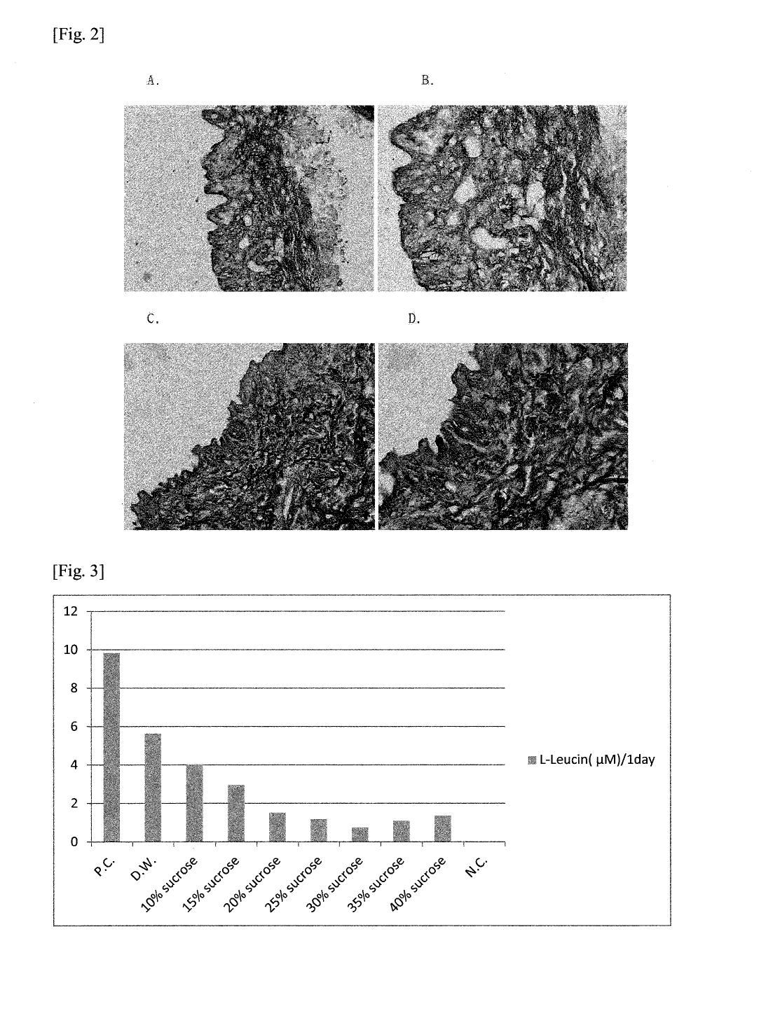

The optical microscope photographs and scanning electron microscope photographs of the skins of Example and Comparative Example are represented in FIGS. 1 and 2.

From the results of FIGS. 1 and 2, it can be known that Example shows excellent structural stability of collagen—which consists of dermis in the tissue, compared with Comparative Example.

In addition, from the microscope photographs of FIGS. 1 and 2, it can be known that in Example the destruction of tissue at a freezing step is remarkably reduced as compared with Comparative Example.

That is, the processing method of the present invention provides high stability of tissue compared with the conventional freeze-drying method. As a result, an acellular dermal matrix according to the present processing method can increase the success rate of grafting and curtail the treatment duration.

### Experimental Example 2

### Measurement of Degradability by Collagenase

To evaluate the stability of acellular dermal matrix according to the concentration of sucrose, the degradability by collagenase was measured as follows:

(1) 25 mg of sample was added to 5 mM TES buffer containing 0.36 mM calcium chloride and mixed well.

(2) 0.1 ml of collagenase (0.1 mg/ml) was added to the sample of step (1) and incubated at 37° C. for one day with stirring.

(3) 4.0 mM L-leucine standard solution was serially diluted and treated with ninhydrin color reagent. A standard curve was prepared by measuring absorbance at 570 nm (VERSA max, Molecular Device, USA).

(4) The sample of step (2) was treated with ninhydrin color reagent and then absorbance at 570 nm was measured.

(5) The amount of released L-leucine from each sample was calculated by using the L-leucine standard curve of step (3).

The above calculated L-leucine release amount is represented in FIG. 3. As can be seen from FIG. 3, a cryopreserved acellular dermal matrix—which is processed with a cryoprotectant comprising 20 to 40% by weight of sucrose in the final concentration—shows high stability of tissue so that degradation rate by collagenase is remarkably reduced.

1. A method for preparing a cryopreserved acellular dermal matrix comprising:
i) removing epidermis of allograft skin;
ii) removing cells in dermis;
iii) mixing glycerol, and a basic solution selected from a buffer solution and an animal cell culture medium;
iv) dissolving sucrose in the solution to a final concentration of 20 to 40% by weight to obtain a cryoprotectant;
v) penetrating the cryoprotectant into the skin from which epidermis and cells in dermis are removed; and
vi) freezing the cryoprotectant-penetrated skin in a controlled rate freezer.

2. The method for preparing a cryopreserved acellular dermal matrix according to claim 1, wherein the mixing ratio of glycerol and the basic solution is 0.5˜3:9 based on weight.

3. The method for preparing a cryopreserved acellular dermal matrix according to claim 1, wherein the buffer solution is selected from PBS (phosphate buffered saline), TBS (Tris-buffered saline) and citric acid buffer.

4. The method for preparing a cryopreserved acellular dermal matrix according to claim 1, wherein the animal cell culture medium is selected from MEM (Minimum Essential Media), DMEM (Dulbecco's Modified Eagle Media), RPMI 1640, IMDM (Iscove's Modified Dulbecco's Media), Defined Keratinocyte-SFM (without BPE), Keratinocyte-SFN (with BPE), KnockOut D-MEM, AmnioMAX-II Complete Medium and AmnioMAX-C100 Complete Medium.

5. The method for preparing a cryopreserved acellular dermal matrix according to claim 1, wherein the final concentration of sucrose is 30% by weight.

6. The method for preparing a cryopreserved acellular dermal matrix according to claim 1, wherein the cryoprotectant is penetrated into the separated skin in a 4° C. low-temperature bath for 6 to 24 hours.

7. The method for preparing a cryopreserved acellular dermal matrix according to claim 1, wherein the cryoprotectant-penetrated skin is frozen in a controlled rate freezer at a freezing rate of −1° C. per minute.

8. An autograft substitute comprising the cryopreserved acellular dermal matrix which is prepared by the method according to any one of claims 1 to 7.
